(19) United States
(12) Patent Application Publication
(10) Pub. No.: US 2017/0185732 A1
(43) Pub. Date: Jun. 29, 2017

(54) PATIENT MONITORING SYSTEM WITH NETWORK OF TREATMENT EQUIPMENT

(71) Applicant: Ethicon Endo-Surgery, Inc., Cincinnati, OH (US)

(72) Inventors: Paul J. Niklewski, Cincinnati, OH (US); James F. Martin, Lebanon, OH (US); Ross G. Krogh, Long Grove, IL (US); Gregory D. Bishop, Hamilton, OH (US); Donn C. Mueller, Chapel Hill, NC (US)

(21) Appl. No.: 14/982,185

(22) Filed: Dec. 29, 2015

(51) Int. Cl. G06F 19/00; A61M 16/00; A61B 5/00; A61M 5/14; A61B 5/145; A61B 5/021

(52) U.S. Cl. CPC G06F 19/3406; G06F 19/3456; A61B 5/14542; A61B 5/021; A61B 5/4821; A61M 5/14; A61M 16/00

(57) ABSTRACT

A patient preparing for a medical procedure is fitted with a modular connector device that stores patient data, medical procedure data, and other software. The patient connector can then be connected to other modular devices via wired or wireless means, allowing the other modular devices to access the patient connector storage to immediately identify the patient and determine its role within the medical procedure. The patient connector and each modular device may connect wirelessly to a localized network, allowing each device to communicate with each other or with a network server in order to receive updated information on a patient or medical procedure, or to download and configure new device drivers when two incompatible devices attempt to connect. In such a network, a patient may move seamlessly from room to room or from device to device without time consuming manual configurations.

### BACKGROUND

Patient monitoring systems may be used to monitor physiological parameters of patients undergoing diagnostic procedures, surgical procedures, and/or various other types of medical procedures. In some settings, a nurse or technician in a pre-procedure room may prepare a patient for an upcoming procedure. This preparation may include connecting, monitors to the patient for the purpose of obtaining baseline data to be used in the procedure. Such monitors may include a blood pressure monitor and pulse oximetry monitor, among others. Blood pressure readings may be taken by a blood pressure cuff, whereby a nurse or technician secures the cuff around a patient's arm and uses a device to pump air into the cuff. Once the reading from the cuff stabilizes. the nurse or technician may have to manually record the data (e.g., handwritten on a sheet of paper or typed into a portable electronic device), and save this information for later reference during the procedure and eventually, for a patient report. For the nurse or technician to take a pulse oximeter reading, he or she may have to boot up the pulse oximeter module, secure a pulse oximeter probe upon the patient, and take a reading of the patient. Thiss reading may &so be written down on paper or otherwise be manually recorded for later use. Once it is determined the patient is ready for the procedure, the nurse or technician may have to disengage the blood pressure cuff and pulse oximetry probes from the patient, so the patient can be transported from the pre-procedure room to the procedure room.

After the patient enters the procedure room and before the procedure begins, several tasks may be needed to prepare the patient for the procedure. The nurse or technician may have to reconnect both blood pressure and pulse oximetry readers before the procedure can begin. In addition to blood pressure and pulse oximetry, other connections such as, for example, capnography, supplemental oxygen, and electrocardiogram may be required. A great deal of time may be required to connect the physiological monitors to the patient and to connect the physiological monitors to the monitoring system. In some such instances, the nurse or technician must spend time reconnecting the same kinds of physiological monitors that were previously connected to the patient in the pre-procedure room. The time it takes to make these connections may occupy valuable procedure room time, thus decreasing practice efficiency.

In various settings, it may also be desirable to deliver drugs to a patient during a procedure, such as via an IV and/or face mask, etc. Such drugs may include sedatives, anelgesics, amnestics, etc. In some instances, such drugs may be selected and/or combined to place a patient in a state of “conscious sedation” (in lieu of simply rendering a patient completely unconscious through a general anesthetic). Certain systems may also be used to automate the delivery of such drugs. For instance, such systems may be located in the same room where a medical procedure is performed, and may be coupled with a physiological monitoring system to automatically tailor the delivery of drugs based on patient parameters detected by the monitoring system. Examples of such systems are disclosed in U.S. Pat. No. 6,745,764, entitled “Apparatus and Method for Providing a Conscious Patient Relief from Pain and Anxiety Associated with Medical or Surgical Procedures,” issued Jun. 8, 2004, the disclosure of which is incorporated by reference herein; U.S. Pat. No. 7,833,213, entitled “Patient Monitoring and Drug Delivery System and Method,” issued Nov. 16, 2010, the disclosure of which is incorporated by reference herein; U.S. Pat. No. 7,935,081, entitled “Drug Delivery Cassette and a Medical Effector System,” issued May 3, 2011, the disclosure of which is incorporated by reference herein; U.S. Pub. No. 2009/0292179, entitled “Medical System having a Medical Unit and a Display Monitor,” published Nov. 26, 2009, the disclosure of which is incorporated by reference herein; and U.S. Pub. No. 2010/0010433, entitled “Medical System which Controls Delivery of a Drug,” published Jan. 14, 2010, the disclosure of which is incorporated by reference herein.

In addition to the time necessary to connect, configure, and prepare the various monitors and drug delivery devices before and after procedures, conventional systems may also require significant investment into capital components. For example, in a facility with multiple procedure rooms, each procedure room may have a drug delivery unit even if the procedure presently being performed in a given procedure room does not require a drug delivery unit. This may be due to the drug delivery unit being integrated with other equipment that is in use, or because of the cost and risk of repeatedly detaching and transporting the unit, or for a variety of other reasons. Similarly, due to the time required to attach the cables and configure the software necessary to make a unit available in a specific procedure room, it may not be possible to keep a number of drug delivery units available in a centralized location that can rotate into procedure rooms as needed. The time and difficulty of moving and configuring capital components can also negatively impact a procedure in the event of an equipment failure that requires replacement equipment to be attached and configured (e.g., manually) for a particular procedure and patient.

While a variety of systems have been made and used for monitoring patients and delivering drugs to patients, it is believed that no one prior to the inventor(s) has made or used the technology as described herein.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1 depicts a perspective view of an exemplary patient monitoring and drug delivery system;

FIG. 2 depicts a perspective view of the patient monitoring unit of the system of FIG. 1;

FIG. 3 depicts a perspective view of the drug delivery unit of the system of FIG. 1;

FIG. 4 depicts a block diagrammatic view of the system of FIG. 1 with additional exemplary components;

FIG. 5 depicts a schematic view of an exemplary network of interconnected devices;

FIG. 6 depicts a flowchart of exemplary steps performed when a device of the network of devices of FIG. 5 is introduced into a procedure;

FIG. 7 depicts a flowchart of exemplary steps performed to retrieve data from devices of the network of devices of FIG. 5; and

FIG. 8 depicts a flowchart showing an exemplary device transition during a procedure.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Conscious Sedation System

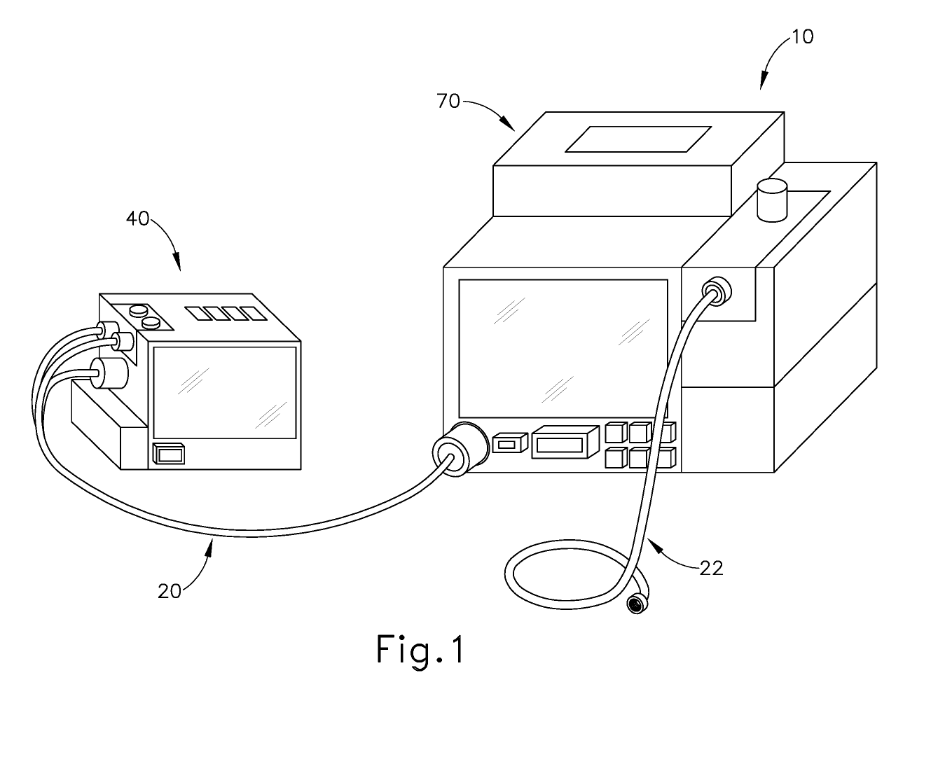

FIG. 1 shows an exemplary patient care system (10) comprising a bedside monitor unit (BMU) (40) and a procedure room unit (PRU) (70). One exemplary use of patient care system (10) is to monitor patient parameters and deliver sedative, analgesic, and/or amnestic drugs to a conscious, non-intubated, spontaneously-ventilating patient undergoing a diagnostic procedure, surgical procedure, or other medical procedure by a physician. This use is not exhaustive of all of the potential uses of the invention but will be used to describe examples herein. BMU (40) and PRU (70) are connected via communication cable (20). Communication cable (20) provides means for transmitting electronic data as well as various hydraulic signals and gases between BMU (40) and PRU (70). For instance, communication cable (20) may include a plurality of pneumatic tubes and a plurality of electrical wires, all integrated within a single sheath or cable. Communication cable (20) may be removed from both BMU (40) and PRU (70) to facilitate practice efficiency and user convenience. BMU (40) and PRU (70) are free to move independently of each other if communication cable (20) is not in place. This allows for mobility of each unit independent of the other; this feature is especially important in hospitals that have a great deal of medical procedures and there is little time to connect patients to monitors. BMU (40) and PRU (70) preferably accommodate an external oxygen source that is intended to provide supplemental oxygen to the patient during the course of a surgical procedure if the clinician so desires. An IV tube set (22) is shown connected to PRU (70) and delivers sedative or amnestic drugs to a patient during a surgical procedure.

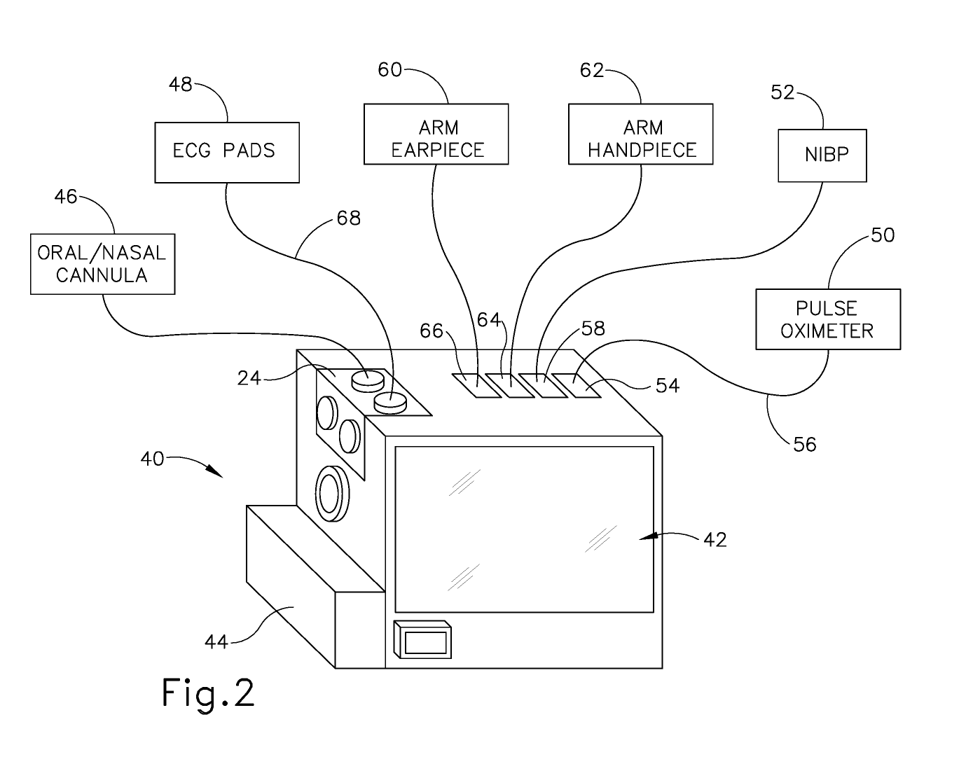

BMU (40) serves as a patient monitoring unit, monitoring various physiological parameters of a patient. As shown in FIG. 2, BMU (40) is compact and portable so it requires relatively little effort to move from one room to another. In some versions, BMU (40) could mount upon either an IV pole or a bedrail; this would free the clinician from the burden of carrying the unit wherever the patient needs to be transported. BMU (40) is small and light enough to be held in the hand of a nurse or technician. BMU (40) allows the user to input information via a touch screen assembly (42) or a simple keypad, etc. Touch screen assembly (42) is provided as an overlay on a display device that is integrated into one surface of BMU (40), and that displays patient and system parameters, and operational status of BMU (40). An exemplary bedside touch screen assembly (42) is a 5.25″ resistive touch screen manufactured by MicroTech mounted upon a 5.25″ color LCD screen manufactured by Samsung. Other suitable forms that a display screen and touch screen may take will be apparent to those of ordinary skill in the art in view of the teachings herein. An attending nurse or physician may enter patient information such as, for example, patient weight and a drug dose profile into BMU (40) by means of bedside touch screen assembly (42). A BMU battery (44) is fixedly attached to the BMU (40) and comprises a standard rechargeable battery such as, for example, Panasonic model no. LC-T122PU, that is capable of supplying sufficient power to run BMU (40) for an extended period of time. In some versions, BMU battery (44) can be recharged while BMU (40) is connected to PRU (70) via communication cable (20) or can be charged directly from an independent power source. Various suitable ways in which battery (44) may be charged will be described in greater detail below in section III.A.; while still other suitable ways will be apparent to those of ordinary skill in the art in view of the teachings herein. Similarly, various suitable forms that battery (44) may take, as well as various suitable compositions thereof, will be apparent to those of ordinary skill in the art in view of the teachings herein.

As shown in FIG. 2, BMU (40) may be connected to a plurality of patient sensors and peripherals used to monitor patient vital signs and deliver supplemental oxygen to the patient. Oral nasal cannula (46) delivers oxygen from an external oxygen source and collects samples of exhaled gas. Oral nasal cannula (46) is removably attached to cable pass-through connection (24). Cable pass-through connection (24) sends the signal obtained by oral nasal cannula (46) directly to a capnometer (e.g., a CardioPulmonary Technologies CO2WFA OEM) in PRU (70) and preferably via communication cable (20) (FIG. 1). The capnometer measures the carbon dioxide levels in a patient's inhalation/exhalation stream via a carbon dioxide-sensor as well as measuring respiration rate. Also attached to the cable pass-through connection (24) is a standard electrocardiogram (ECG) (48), which monitors the electrical activity in a patient's cardiac cycle. The ECG signals are sent to the PRU (70) where the signals are processed. A pulse oximeter probe (50) (e.g., by Dolphin Medical) and a non-invasive blood pressure (NIBP) cuff (52) are also connected to BMU (40) in the present example. Pulse oximeter probe (50) measures a patient's arterial saturation and heart rate via an infrared diffusion sensor. The data retrieved by pulse oximeter probe (50) is relayed to pulse oximeter module (54) (e.g., by Dolphin Medical) by means of pulse oximeter cable (56). The NIBP cuff (52) (e.g., a SunTech Medical Instruments PN 92-0011-00) measures a patient's systolic, diastolic, and mean arterial blood pressure by means of an inflatable cuff and air pump (e.g., by SunTech Medical), also incorporated as needed. NIBP cuff (52) is removably attached to NIBP module (58) located on BMU (40).

In the present example, a patient's level of consciousness is detected by means of an Automated Responsiveness Monitor System (ARM), though like various other components described herein, an ARM system is merely optional and is not required. An exemplary ARM system is disclosed in U.S. Pub. No. 2005/0070823, entitled “Response Testing for Conscious Sedation Involving Hand Grip Dynamics,” published Mar. 31, 2005, the disclosure of which is incorporated by reference herein. The ARM system of the present example comprises a query initiate device and a query response device. The ARM system operates by obtaining the patient's attention with the query initiate device and commanding the patient to activate the query response device. The query initiate device may comprise any type of stimulus device such as a speaker via an earpiece (60), which provides an auditory command to a patient to activate the query response device. The query response device of the present example comprises is a handpiece (62) that can take the form of, for example, a toggle or rocker switch or a depressible button or other moveable member hand held or otherwise accessible to the patient so that the member can be moved or depressed by the patient upon the patient's receiving of the auditory signal or other instruction to respond. Alternatively, a vibrating mechanism may be incorporated into handpiece (62) that cues the patient to activate the query response device. For instance, in some versions, the query initiate device comprises a cylindrical handheld device (62), containing a small 12V DC bi-directional motor enabling the handheld device to vibrate the patient's hand to solicit a response.

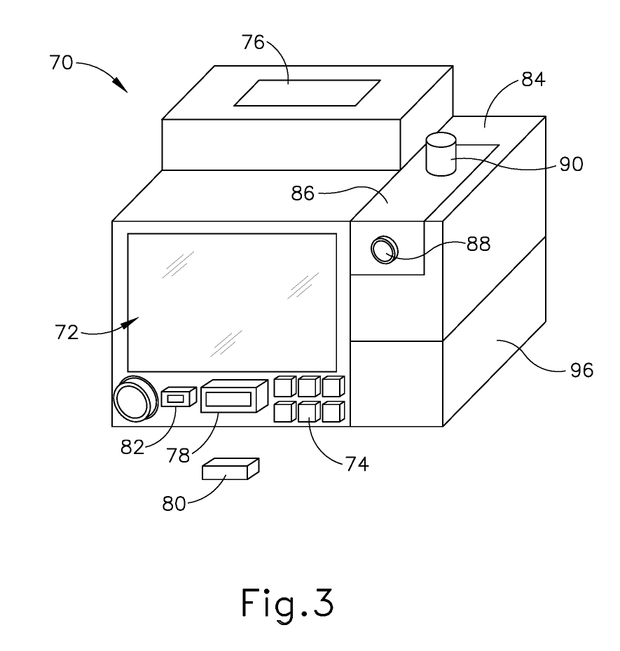

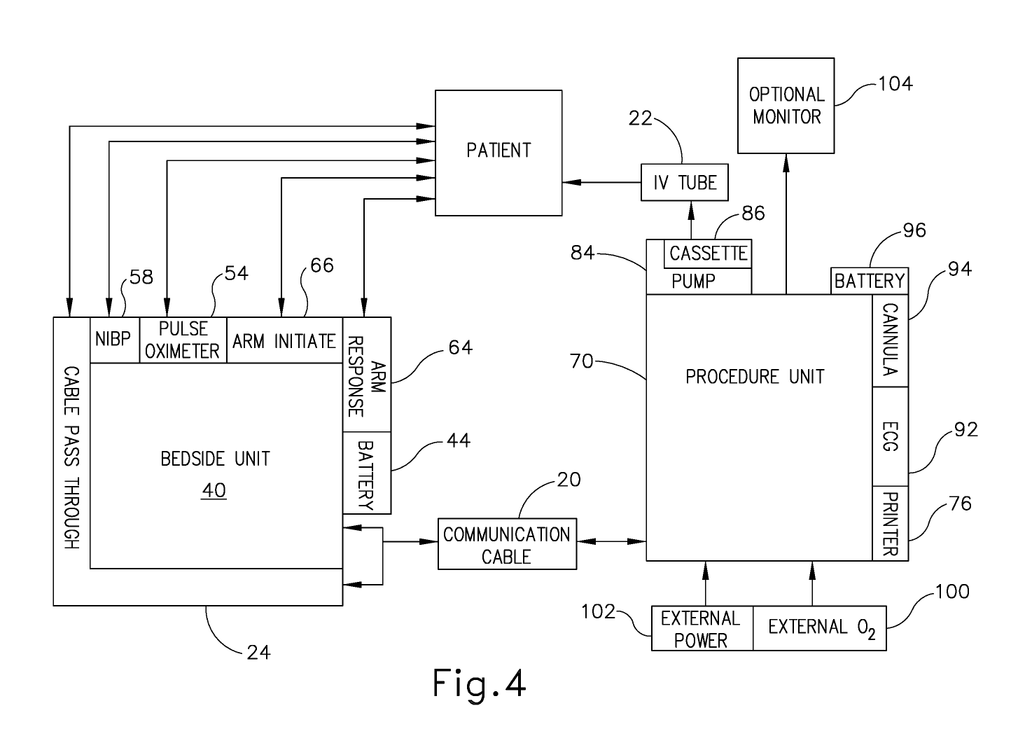

After the query is initiated, the ARM system generates signals to reflect the amount of time it took for the patient to activate the query response device in response to the query initiate device. These signals are processed by a logic board located inside BMU (40) and are displayed upon either bedside touch screen assembly (42), procedure touch screen assembly (72) (FIG. 3), and/or an optional monitor 104 (FIG. 4). The amount of time needed for the patient to respond to the query gives the clinician an idea as to the sedation level of the patient. The ARM system has two modules in this example, including a query response module (64) and a query initiate module (66), collectively referred to as the ARM system modules (64, 66). ARM system modules (64, 66) have all the necessary hardware to operate and connect the query response device (62) and the query initiate device (60) to BMU (40).

In some versions monitoring modules (54, 58, 64, 66) are easily replaceable with other monitoring modules in the event of malfunction or technological advancement. These modules (54, 58, 64, 66) include all of the necessary hardware to operate their respective peripherals. The above-mentioned patient modules (54, 58, 64, 66) are connected to a microprocessor-based electronic controller or computer (MLB) located within each of the PRU (70) and BMU (40). The electronic controller or main logic board comprises a combination of available programmable-type microprocessors and other “chips,” memory devices and logic devices on various board(s) such as, for example, those manufactured by Texas Instruments (e.g., XK21E) and National Semiconductor (e.g., HKL72), among others. Various other suitable forms that modules (54, 58, 64, 66) and associated electronics may take will be apparent to those of ordinary skill in the art in view of the teachings herein.

Once BMU (40) and PRU (70) are connected via communication cable (20), ECG and capnography may be monitored, and supplemental oxygen may be delivered to the patient. It should be understood, however, that these connections may be made in the pre-procedure room to increase practice efficiency. By making these connections in the pre-procedure room, less time may be required in the procedure room connecting capnography, ECG and supplemental oxygen to PRU (70). Oral nasal cannula (46) and ECG leads (68) are connected directly to cable pass-through connection (24). Cable pass-through connection (24), located on BMU (40), is essentially an extension of communication cable (20), which allows the signals from ECG leads (68) and oral nasal cannula (46) to bypass BMU (40) and be transferred directly to PRU (70). It will be evident to those skilled in the art, however, that the BMU (40) could be configured to accept the ECG (48) and oral/nasal cannula (46) signals and process the signals accordingly to provide the information on screen (42) and supplemental oxygen to the patient in the pre-procedure room. Other examples of components, features, and functionality that may be incorporated into BMU (40) will be described in greater detail below; while still further examples of components, features, and functionality that may be incorporated into BMU (40) will be apparent to those of ordinary skill in the art in view of the teachings herein.

Referring now to FIG. 3, PRU (70) allows a physician to safely deliver drugs, such as sedative, analgesic, and/or amnestic drugs to a patient, and monitor the patient during a medical procedure. Procedure touch screen assembly (72) comprises a display device that is integrated into the surface of PRU (70), which displays patient and system parameters, and operation status of PRU (70). In some versions, procedure touch screen assembly (72) comprises a 15″ resistive touch screen manufactured by MicroTech mounted upon a 15″ color LCD screen manufactured by Samsung. Other suitable forms that a display screen and touch screen may take will be apparent to those of ordinary skill in the art in view of the teachings herein. It should be noted that, in the present example, procedure touch screen assembly (72) is the primary display and user input means, and is significantly larger than the bedside touch screen assembly (42) and is capable of displaying more detailed information. In addition to procedure touch screen assembly (72), the user may input information into PRU (70) by means of drug delivery controls (74). Drug delivery controls (74), such as buttons, dials, etc., are located on one side of PRU (70) and allow the clinician to change various system parameters and bypass procedure touch screen assembly (72). A printer (76) is integrally attached to the top of PRU (70). Printer (76) allows the clinician to print a patient report that includes patient data for pre-op and the procedure itself. The combination of printing a patient report and the automatic data logging features may decrease the amount of time and effort a nurse or technician must spend regarding patient condition during the course of a procedure. Printer (76) receives data signals from a printer interface (e.g., Parallel Systems CK205HS), which is located on the main logic board. Printer (76) may comprise a thermal printer (e.g., Advanced Printing Systems (APS) ELM 205HS) and/or any other suitable type of printer. It should also be understood that printer (76) may be remote from PRU (70) and may even be omitted altogether, if desired.

Memory card reader (78), which includes a slot in the outer casing of PRU (70), allows flash memory card (80) to be inserted and removed from PRU (70). Flash memory card (80) is a solid-state storage device used for easy and fast information storage of the data log generated by PRU (70). The data is stored so that it may be retrieved from flash memory card (80) at a later time. In some versions, memory card reader (78) accepts flash memory card (80) containing software to upgrade the functionality of patient care system (10). Again, as with other components described herein, memory card reader (78) may be modified, substituted, supplemented, or omitted as desired. In the present example, memory card reader (78) is supplemented with a data port (82). Data port (82) may include, but is not limited to, a standard serial port, a USB port, a RS232 port, an Ethernet port, or a wireless adapter (e.g., using IEEE 802.11n/g/b/a standard, etc.). Data port (82) may be used to link PRU (70) to an external printer to print a patient report or to transfer electronic files to a personal computer or mainframe. A merely illustrative example of how data port (82) may be used to communicate with a centralized network system component will be described in greater detail below in section III. B., while still other suitable examples will be apparent to those of ordinary skill in the art in view of the teachings herein.

PRU (70) delivers fluid to a patient via an infusion pump, such as a peristaltic infusion pump (84) (e.g., by B-Braun McGaw). Peristaltic infusion pump (84) is integrally attached to PRU (70), and uses peristaltic fingers to create a wavelike motion to induce fluid flow inside a flexible tube connected to a fluid reservoir. A drug cassette (86) is a generally rectangular shaped structure that is placed adjacent to peristaltic infusion pump (84). Drug cassette (86) of this example is made of a rigid thermoplastic such as, for example, polycarbonate. Drug cassette (86) has an internal cavity that houses IV tubing (22) made of a flexible thermoplastic such as, for example, polypropylene (e.g., Kelcourt). Drug cassette (86) receives tubing (22) via a port (88) and accurately and reliably positions exposed IV tubing (22) in contact with the peristaltic fingers of peristaltic infusion pump (84). IV tube set (22) attaches to a fluid vial (90), and a portion of the length of IV tube set (22) is contained within drug cassette (86). Another portion of IV tube set (22) lies external to drug cassette (86) to facilitate the interaction with peristaltic pump (84). IV tubing (22) is coiled within drug cassette (86) and has a length to reach a patient removed from the PRU (70). A fluid detection sensor (not shown) may be mounted to an inner wall of drug cassette (86). Such a fluid detection sensor may comprise any one of known fluid sensors, such as the MTI-2000 Fotonic Sensor, or the Microtrak-II CCD Laser Triangulation Sensor both by MTI Instruments Inc. IV tube set (22) may run through the fluid detection sensor before exiting drug cassette (86). PRU (70) may include features operable to prime IV tubing (22) with relative ease for a user. Various examples of how such priming may be provided are disclosed in U.S. Pat. No. 7,833,213, the disclosure of which is incorporated by reference herein.

In the present example, drug cassette (86) includes just one vial (90). However, it should be understood that some versions of drug cassette (86) may include several vials (90). Such vials (90) may include the same drug. Alternatively, a plurality of vials (90) associated with a single drug cassette (86) may include a variety of different kinds of drugs. In other words, a single drug cassette (86) may be used to selectively deliver two or more drugs simultaneously and/or in a particular sequence. While vials (90) are used in the present example, it should be understood that any other suitable type of container may be used as will be understood by those of ordinary skill in the art in view of the teachings herein. It should also be understood that some versions of PRU (70) may be configured to receive two or more drug cassettes (86). Each such drug cassette (86) may be associated with a single drug (e.g., different drug cassettes (86) used for different drugs), or each drug cassette (86) may be associated with a combination of drugs (e.g., different drug cassettes (86) used for different combinations of drugs).

FIG. 4 shows how components of system (10) interface with each other and with a patient. While not shown in FIG. 3, FIG. 4 shows how PRU (70) includes an integral ECG module (92) and integral cannula module (94). ECG module (92) is coupled with ECG (48) via ECG leads (68) extending from pass-through connection (24). Cannula module (94) is coupled with oral/nasal cannula (46), also through pass-through connection (24). Like modules (54, 58, 64, 66) described above, modules (92, 94) may be easily replaceable with other monitoring modules in the event of malfunction or technological advancement. Modules (92, 94) may also include all of the necessary hardware to operate their respective peripherals, and may be further coupled with a microprocessor-based electronic controller or computer located within PRU (70) and/or BMU (40).

As also shown in FIG. 4, PRU (70) of the present example is coupled with an external oxygen source (100), an external power source (102), and an external monitor (104). External oxygen source (100) may by regulated by one or more components of PRU (70), which may deliver oxygen from oxygen source (100) to the patient based on one or more parameters sensed by BMU (40), based on drug delivery from cassette (86), and/or based on other factors. External power source (102) may be used as a primary source of power for PRU (70), with a battery (96) being used as a backup power source. Alternatively, battery (96) may be used as a primary source of power for PRU, with external power source (102) being used for backup power and/or to charge battery (96). External monitor (104) may be used to supplement or to substitute the display features of touch screen assembly (42) and/or touch screen assembly (72). For instance, external monitor (104) may display information including patient physiological parameters, status of operation of system (10), warning alerts, etc. PRU (70) and/or BMU (40) may communicate with external monitor (104) via cable, wirelessly (e.g., via RF transmission, etc.), or otherwise. Other examples of components, features, and functionality that may be incorporated into PRU (70) will be described in greater detail below; while still further examples of components, features, and functionality that may be incorporated into PRU (70) will be apparent to those of ordinary skill in the art in view of the teachings herein.

II. Exemplary Network of Interconnected Devices Used in Conjunction with Conscious Sedation System

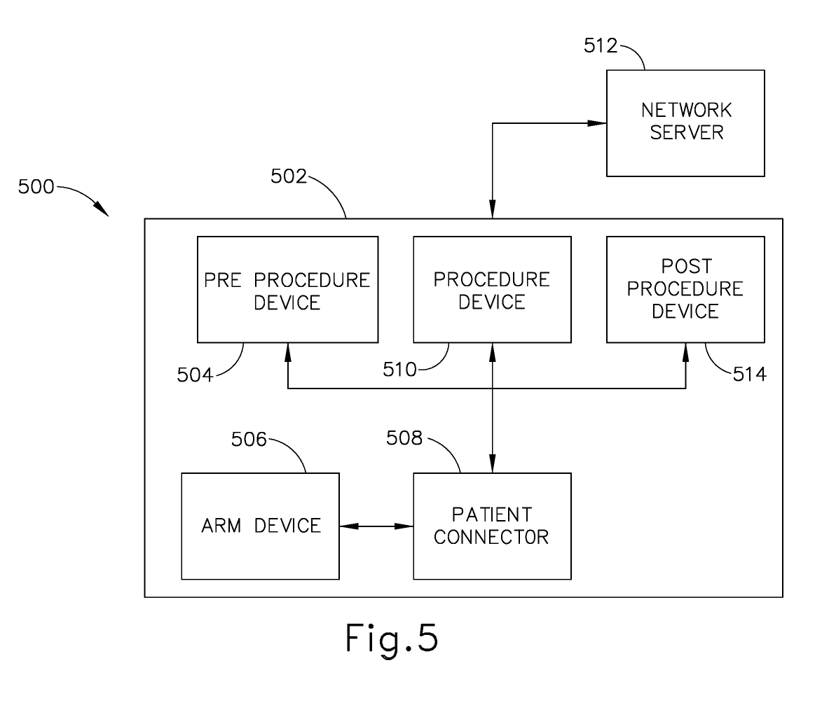

FIG. 5 depicts a schematic view of an exemplary network of interconnected devices (504, 506, 510, 514). In the present example, the interconnected devices (504, 506, 510, 514) operate within a device network (500) having a patient connector (508) serving as a communication hub for a variety of other devices including, for example, an automated responsiveness monitor (506), a set of pre procedure devices (504), a set of procedure devices (510), and a set of post procedure devices (510). Automated responsiveness monitor (506) may be configured in accordance with the ARM described above and/or in any other suitable fashion. Pre procedure devices (504) may include any kinds of devices that tend to be used before a surgical procedure, including but not limited to monitors and sensors, drug or gas delivery devices, such as electrocardiograms, pre-procedure medications including antibiotics/antiemetics, and an ultrasound pre-procedure assessment. Procedure devices (510) may include any kinds of devices that tend to be used during a surgical procedure, including but not limited to ancillary infusion pumps, diagnostic electrocardiogram, and echocardiograms. Post procedure devices (514) may include any kinds of devices that tend to be used after a surgical procedure, including but not limited to patient monitoring, delivery of antiemetics, infusion pumps for post procedural pain management, and post procedural oxygen delivery. Other suitable forms that devices (504, 506, 510, 514) may take will be apparent to those of ordinary skill in the art in view of the teachings herein.

Device network (500) may comprise a wireless or wired local area network, a peer to peer network of devices, a spoke-hub network of devices, or similar types of network topology functioning individually or in combination. Communication across device network (500) may be achieved in varying ways depending upon the particular devices the network is composed of, but may include communication over Wi-Fi, Bluetooth, IR, radio, RFID, NFC, optical, Ethernet, USB, or similar connections.

Patient connector (508) may comprise a computer such as a laptop, a tablet or mobile device, a microcomputer or single board computer, a data storage device, or a similar device having the capability to receive, and transmit data from a variety of input sources. Patient connector (508) may also be comprised of a plurality of distinct devices or may be embodied by a single device. It may be desirable for patient connector (508) to be easily movable so that it may travel with a patient as a patient moves from one room to another after admission for medical care. Thus, in some versions, patient connector (508) may comprise a laptop computer or tablet that is mounted to a bed or chair that moves with the patient. Similarly, in other versions, patient connector (508) may be integrated with a device worn by the patient, such as respiratory equipment, intravenous delivery equipment, sensor equipment, or the like. As an example, a patient may, upon admission, be fitted with a respiratory mask or harness and one or more sensors attached to their head, neck, chest, or other extremities. Patient connector (508) may be integrated with a harness or mask that the patient will be wearing during treatment, and may be capable of wireless or wired communication with devices and sensors that the harness or mask is used with. In this manner, when the respirator mask is attached to a device providing oxygen and/or measuring exhaled gasses (e.g., a capnometer), patient connector (508) may also be connected to the respirator device.

In some versions, patient connector (508) may in some embodiments store a variety of data and information. For example, patient connector (508) may be configured to store the identity of the patient it is currently associated with, electronic medical records for the patient, information relating to past, present, or future procedures associated with the patient, software drivers for systems and devices associated with the procedures, and other information, data, or software.

Patient connector (508) may also be configured to have the capability to provide a patient identification to another device within device network (500), provide patient medical information such as drug allergies or current medication to another device within device network (500), provide medical procedure information, including drug delivery rates and thresholds and other automated activity configurations and thresholds to device network (500), or provide software and drivers to enable a first device of device network (500) to communicate or function with a second device of device network (500). Patient connector (508) may also be in communication with a network server (512), and may allow one or more devices of device network (500) to communicate with network server (512) directly or indirectly if they do not otherwise have the capability to do so.

Network server (512) may comprise a remotely located server or plurality of servers having access to information, data, and software that could be transmitted to patient connector (508). For example, patient connector (508) may be initially configured for a particular patient and procedure, and, upon being configured, would receive data and drivers from network server (512) appropriate for that particular patient and procedure and for the devices that may be involved. Patient connector (508) may then distribute data or drivers to a device of device network (500). Additionally, devices of device network (500) that have the capability to communicate with network server (512) directly may receive the same data or a subset of data that patient connector (508) receives, to allow for redundant availability of the data in the event that a particular device must be added or removed from device network (500); or in the event that network server (512) becomes unreachable during a procedure.

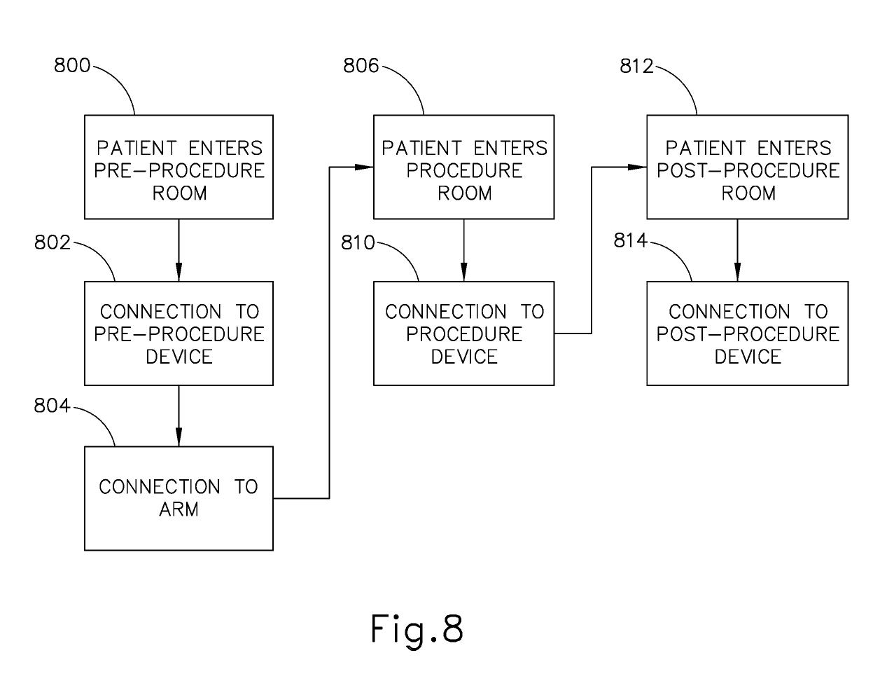

One capability allowed by the infrastructure shown in FIG. 5 and described above is for a patient to transition from one set of devices (504, 506, 510, 514) or a procedure room to another set of devices (504, 506, 510, 514) or procedure room without requiring time consuming and error prone manual configuration of devices (504, 506, 510, 514) FIG. 8 shows an exemplary set of steps that may be used to establish connections with respect to FIG. 5 during the stages of before, during, and after a surgical procedure. For example, a patient may be initially equipped with or proximate to a patient connector (508) that is configured with the appropriate data for the patient, the procedure, and the devices (504, 506, 510, 514) associated with the procedure. A patient with a patient connector (508) may enter a pre-procedure room (block 800). Upon entering the pre-procedure room (block 800), patient connector (508) may automatically establish one or more connections (block 802) with pre-procedure devices (504) based upon a wireless exchange between pre procedure devices (504) or the connection of one or more data cables. The patient may also be connected with an automated responsiveness monitor device (506), which may be attached to the patient and a wireless connection may be established (block 804) between ARM device (506) and patient connector (508). ARM device (506) may receive patient and procedure specific configurations from patient connector (508), establishing the types and sequence of stimuli that ARM device (506) will produce at various times; and may also receive a software driver allowing ARM device (506) to communicate with a BMU (40) or PRU (70) that is likely to be used during the procedure. As with ARM (506), pre-procedure devices (504) may connect wirelessly, or in some cases via a physical connection, to patient connector (508) and receive data, configurations, and drivers as needed.

Once the pre-procedures are complete, the patient may be moved to a procedure room (block 806). Pre procedure devices (504) may, through a physical disconnection of a cable or by a wireless proximity, disconnect from patient connector (508). As the patient arrives in the procedure room (block 806), for example a surgical suite, a set of procedure devices (510) may automatically connect and be configured as described above (block 810). After the medical procedure (e.g., surgery) is complete, as the patient exits the procedure room and transitions to a post procedure room (block 812), procedure devices (510) may automatically disconnect from patient connector (508) and post procedure devices (514) may automatically connect (block 814) with patient connector (508). In this manner, sets of devices (504, 506, 510, 514) may be quickly brought online and configured for a particular patient and procedure without manual intervention, while at the same time different devices such as the ARM (506) may travel with the patient and stay connected throughout.

Similarly, this capability allows for a single device (504, 506, 510, 514) to be added or removed in the event of a device failure or unexpected need. For example, suppose that during a post-procedure stage, a set of post procedure devices (514) are configured and in communication with patient connector (508). For instance, a PRU (70) may fail, requiring replacement. A clinician could disconnect PRU (70) and power it down, causing it to disconnect from patient connector (508). A new PRU (70) could then be placed in the room and powered on, and connected via cables or wireless connection to patient connector (508). Patient connector (508) could provide locally available data to the PRU (70), thereby configuring it identically to the previous PRU (70) and making it immediately available for the post procedure monitoring or treatment. In this manner, the period where a properly configured PRU (70) is unavailable is minimized, because a clinician does not need to manually configure any delivery limits, monitoring alarms, or similar capabilities for the particular patient and procedure; and compatibility with other post procedure devices (514) can be ensured due to the device drivers or indirect communication provided by patient connector (508).

In the case of a multi component patient connector (508), each device (504, 506, 510, 514) within the device network (500) may have one or more components allowing it to function individually or in the aggregate as a patient connector (500). For example, in some versions, each device (504, 506, 510, 514) within device network (500) could store similar sets of data and drivers from network server (512) and could be capable of communication with other devices (504, 506, 510, 514) within network (500). In this manner, the overall device network (500) can survive the failure of multiple devices (504, 506, 510, 514), including network server (512), while still maintaining the ability to automatically configure replacement devices (504, 506, 510, 514).

III. Exemplary Methods of Managing Interconnected Devices

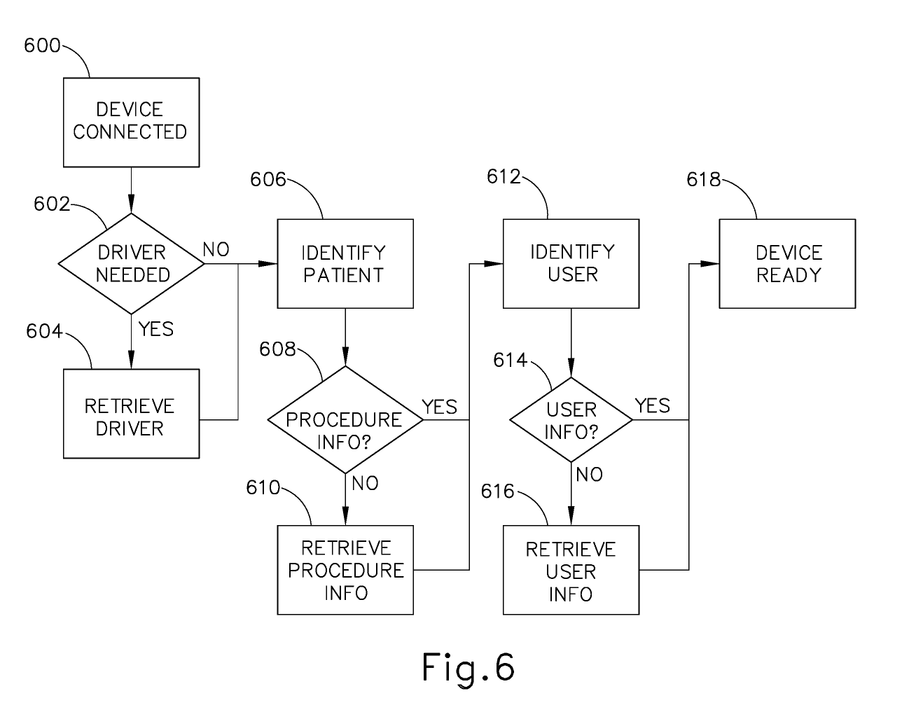

FIG. 6 depicts a flowchart of exemplary steps that may be performed when a device (504, 506, 510, 514) is introduced into a procedure using the arrangement shown in FIG. 5. With a patient and an associated patient connector (508) being configured and present with device network (500), the steps of FIG. 6 may be performed any time that an additional device (504, 506, 510, 514) is connected to patient connector (508) via device network (500). This could include a single device (504, 506, 510, 514) being connected to a patient connector (508), such as when an additional device (504, 506, 510, 514) is needed or a malfunctioning device (504, 506, 510, 514) is replaced; but could also include multiple devices (504, 506, 510, 514) being connected, such as when a patient is moved from a pre-procedure room to a procedure room, and procedure devices (510) of the procedure room are connected to patient connector (508).

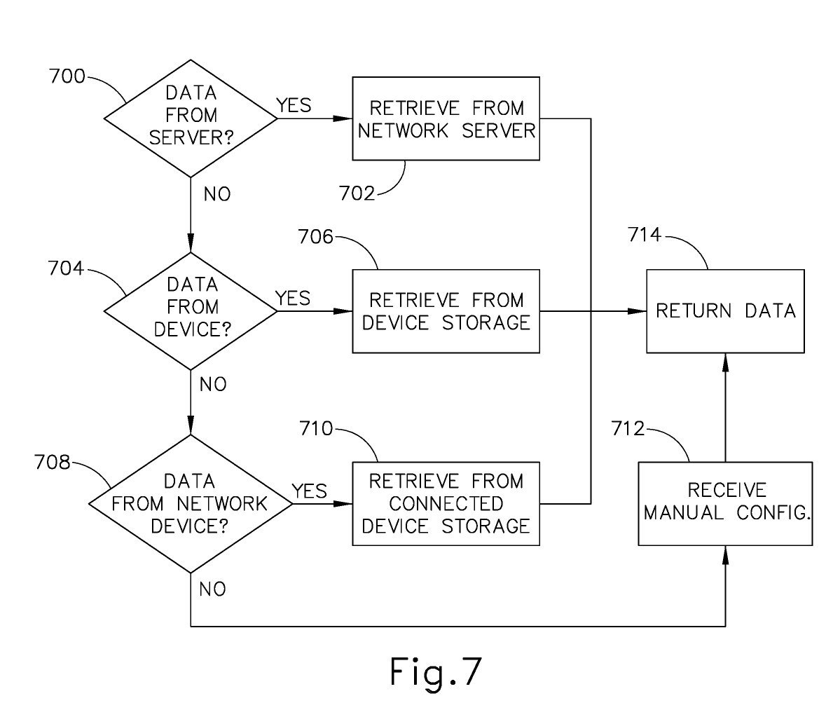

When the new device (504, 506, 510, 514) is connected (block 600) to patient connector (508), patient connector (508) or the new device (504, 506, 510, 514) will determine if drivers are needed (block 602). Drivers may be required to allow the new device (504, 506, 510, 514) to communicate with one or more other devices (504, 506, 510, 514) within device network (500). For example, a particular BMU (40) may require an additional driver before it can function with a PRU (70), especially where BMU (40) and PRU (70) originate from different manufacturers, have recently been put into service, or have been in service for a significant period of time without software updates. If a driver is needed in order for the new device (504, 506, 510, 514) to function with one or more others devices (504, 506, 510, 514) of device network (500), the new device (504, 506, 510, 514) will retrieve the required drivers (block 604). Retrieving drivers (block 604), retrieving medical procedure information (block 610), and retrieving user information (block 614) via device network (500) may be accomplished according to the steps of FIG. 7, which will be discussed in more detail below.

If it is determined that drivers are not needed (block 602), or after drivers have been retrieved (block 604) and configured on the new device (504, 506, 510, 514), the new device (504, 506, 510, 514) may identify the patient (block 606) that is currently associated with patient connector (508). Patient identification (block 606) may be accomplished by, for example, a wireless or wired communication with patient connector (508), such as a proximity based RFID, Bluetooth, or Wi-Fi signal being exchanged by patient connector (508) and the new device (504, 506, 510, 514), a magnetic or NFC transfer between the devices, or a physical connection of a data or power cable between the devices. Once the patient has been identified (block 606), the new device (504, 506, 510, 514) may determine if it has all required medical procedure information (block 608) for the patient; and if medical procedure information is missing, may attempt to retrieve (block 610) medical procedure information. Medical procedure information may include a variety of characteristics associated with a particular patient and procedure, for example, a patient's height, weight, and other physical characteristics, drug delivery limitations, alarm thresholds, and similar configurations.

Once retrieved (block 610), medical procedure information may be used to configure the new device (504, 506, 510, 514) in an appropriate manner so that the new device (504, 506, 510, 514) can be used appropriately with the patient during the procedure. A user may also be identified (block 612), such as one or more clinicians or staff that will be working with the new device (504, 506, 510, 514) during the medical procedure. User identification (block 612) may be accomplished by a wireless or wired communication between the new device (504, 506, 510, 514) and patient connector (508), or between the new device (504, 506, 510, 514) and another device of device network (500). Once user have been identified (block 612), the new device (504, 506, 510, 514) may determine if required user information is available (block 614), and where user information is not available it may be retrieved (block 616).

User information may include pictures and descriptions of staff involved in a medical procedure, security challenges or verifications for staff involved in the medical procedure to prevent unauthorized use or access of the new device (504, 506, 510, 514), user specific configurations of the new device (504, 506, 510, 514) such as, for example, where a particular user or team of users prefers a particular monitor to emit an audible alarm versus a visual alarm, or where a particular user or team of users prefers a particular display to display certain information organized in certain ways. Such user specific configurations could be configured manually beforehand, or could be saved and updated for a particular device (504, 506, 510, 514) each time device (504, 506, 510, 514) is used within device network (500), so that users of devices can expect a consistent configuration and experience with devices (504, 506, 510, 514). Once user information is retrieved (block 616), it can be used to configure the new device (504, 506, 510, 514) with any user specific settings, preferences, information, or security procedures. After the new device (504, 506, 510, 514) has verified that all required drivers, medical procedure information, or user information is presently available to the new device (504, 506, 510, 514) or has been retrieved by the new device (504, 506, 510, 514), device (504, 506, 510, 514) is fully configured and ready (block 618) for use with the patient and procedure.

As an example of how this process might occur, a BMU (40) may fail while a procedure is being performed. A new BMU (40) is immediately brought into the room, and a data cable from patient connector (508) is attached (block 600) the new BMU (40). Patient connector (508) determines the compatibility of the new BMU (40) with other devices within device network (500), and determines whether the new BMU (40) needs additional software drivers (block 602). Patient connector (508) determines that the new BMU (40) needs an additional software driver to communicate with PRU (70). Patient connector (508) retrieves the driver (block 604) and provides it to the BMU (40), which may then install and configure the driver. Patient connector (508) will identify the patient (block 606) and current procedure for that patient, and then determine whether the new BMU (40) needs additional medical procedure information (block 608) before it may be used in the medical procedure. Patient connector (508) determines that the new BMU (40) needs to be configured with specific alarm limits based upon the patient's height, weight and age. Patient connector (508) retrieves the procedure info (block 610) and provides it to the new BMU (40), which may then be configured for the patient and procedure based upon the provided information.

Patient connector (508) then identifies the user or team of users that will be using the new BMU (40), and whether the new BMU (40) needs any configuration or information (block 614) based upon the identified user (block 612). Patient connector (508) determines that a first user will be using the new BMU (40), and that the new BMU (40) needs to be configured (block 614) to allow the first user access to the new BMU (40) based upon a password or keycard. Patient connector (508) retrieves security information for the first user (block 616) and provides it to the new BMU (40), which may then be configured for the first user's security settings. The new BMU (40) is now fully configured and ready (block 618) to be used for the medical procedure in device network (500).

FIG. 7 depicts a flowchart of exemplary steps that may be performed to retrieve data within device network (500). These steps may be performed by patient connector (508) or another device (504, 506, 510, 514) within device network (500) when it is determined that a device driver, a set of medical procedure information, or a set of user information may be needed by a device (504, 506, 510, 514) that has recently become associated with patient connector (508). The method of the present example begins with an evaluation (block 700) of whether the needed data is on network server (512). If the needed data is available from network server (512), the information will be retrieved from network server (block 702), as network server (512) may generally by the most reliable source of information. The retrieved information may be returned to the requestor (block 714).

There may be instances where needed data is not available from network server (512), such as when there is a network outage preventing communication between device network (500) and network server (512); or network server (512) is malfunctioning or is otherwise unavailable. Where data is not available from the server (block 700), patient connector (508) or another device may check for data on a local storage device (block 704). If the data is available locally, may retrieve the data from storage (block 706). Locally available data may be stored on a storage device in patient connector (508) or the newly added device (504, 506, 510, 514). Such a local storage device may comprise a flash drive, flash memory, memory card, hard drive, or other similar storage type. Where the data is available on a local storage of patient connector (508) or the new device (504, 506, 510, 514), the data may be retrieved from the local storage (block 706) and returned to the requestor (block 714).

In instances where data is not available from a local source (block 704), patient connector (508) or another device may search (block 708) for data amongst all other devices within device network (500) and, where the data is available from a connected device (504, 506, 510, 514), retrieve the needed data from the connected device (block 710) and return the data to the requester (block 714).

Where the data cannot be found from any source, patient connector (508) or the newly added device (504, 506, 510, 514) may notify a user that a manual configuration (712) may be necessary.

As a functional example using the newly added BMU (40) described above in the context of FIG. 6, suppose that the new BMU (40) needs a set of medical procedure data for the particular patient and medical procedure that the new BMU (40) is being added to. Patient connector (508) or the new BMU (40) will first check to see if the data is available (block 700) on network server (512). Patient connector (508) and the new BMU (40) may, in some versions, each have a wireless communication capability that will allow them to contact network server (512) and retrieve (block 702) needed data when the new BMU (40) is added to device network (500). If the data is successfully retrieved from network server (block 702), the data will be returned by patient connector (508) to the new BMU (40); or may be returned by a data retrieval process of the new BMU (40) to a data configuration process of the new BMU (40), depending upon which device successfully retrieves the data. Once the data is available to the new BMU (40), the medical procedure information may be used to configure one or more alarm limits of the new BMU (40) based upon the patient's height, weight, age, and the medical procedure being performed.

If patient connector (508) or the new BMU (40) is unable to find the needed data on the network server (block 700), the devices may search for the data locally (block 704) and retrieve it from a local storage device (block 706) to be returned to the requesting device or process. The local storage for patient connector (508) may comprise for example, a digital memory card that can be pre-loaded with procedure data, drivers, patient data, and the like, and connected to patient connector (508) when a patient is first associated with patient connector (508). The local storage for the new BMU (40) may comprise, for example, an internal storage device configured to retrieve a set of offline data from network server (512) from time to time in order to allow the new BMU (40) to function when network server (512) is unavailable. This set of offline data may function as an offline backup in case of emergencies, and may not affect the configuration of the new BMU (40) until it is retrieved (block 706).

If the needed data is not available from server (block 700) or locally (block 704), patient connector (508) or the new BMU (40) may search other devices within device network (500) for the needed data (block 708). For example, a PRU (70) may store drivers, medical procedure information, or user information on a local storage device that may be accessed and used by PRU (70); but may also be accessed and used by other devices within device network (500) such as patient connector (508) and the new BMU (40). In this manner, in a scenario where network server (512) is offline or unavailable, and the needed data is not stored on patient connector (508) or the new BMU (40), patient connector (508) or the new BMU (40) may identify the needed data on the local storage of the PRU (70) and retrieve the needed data (block 710) so that it can be sent to the new BMU (40) and used to configure the new BMU (40) as needed. If the data is completely unavailable, the new BMU (40) may prompt the user for manual configuration so that the patient's height, weight, and age could be entered manually.

Distribution of data to make it available to devices of device network (500) may be accomplished in different ways. For example, robust and redundant networks may allow a device network (500) to rely heavily upon network server (512) as a source for needed data, meaning that devices (504, 506, 510, 514) of device network (500) may not need to store an abundance of data locally. In other versions, patient connector (508) itself may be relied upon as the primary data source, where patient connector (508) has adequate local storage to securely store a variety of device drivers, medical procedure information, patient information, and user information. In yet other versions, each device (504, 506, 510, 514) within device network (500) may have a local storage, such as a secure digital memory card, that stores a complete set of all data that may needed during a medical procedure to configure devices (504, 506, 510, 514) properly. In this manner, a failure of multiple components would not hamper the system and if even a single device (504, 506, 510, 514) is able to maintain a connection to device network (500), the remaining device (504, 506, 510, 514) would be able to provide the data and configurations needed to bring each replacement device (504, 506, 510, 514) into the medical procedure without manual reconfiguration.

In some versions of the above disclosed technology, patient connector (508) may act as a router for information between two incompatible devices (504, 506, 510, 514) where a device driver is not available. For example, where a BMU (40) and PRU (70) are each able to communicate with and connect to patient connector (508) across device network (500), but are incompatible with each other, and no device driver exists or can be located and retrieved, patient connector (508) can act as a virtual router and receive and route information between BMU (40) and PRU (70). In this manner, although BMU (40) and PRU (70) cannot communicate directly with each other, they may pass information indirectly to each other through patient connector (508), whether it is information on a medical procedure, patient or user, or whether it is operational data such as sensor output.

IV. Exemplary Safety Shell Control for Conscious Sedation System

As previously discussed, medical procedure information may include information such as the physical characteristics of a patient, drug delivery rates, thresholds and alarms, and other such data. Medical procedure information may also include more complex sets of data and algorithms such as, for example, a safety shell control algorithm, which provides drug delivery responses and alerts designed to ensure patient safety during a variety of procedures. Examples of such a safety shell control algorithm are disclosed in U.S. Pat. No. 9,092,559, entitled “Drug Delivery System with Open Architectural Framework,” issued Jul. 28, 2015, the disclosure of which is incorporated by reference herein.

Such drug delivery responses and alert responses may be provided in accordance with a “safety shell” control algorithm executed through a control logic in PRU (70). In some versions, a safety shell provides fully automated drug delivery to the patient from PRU (70), based on conditions detected by BMU (40) and/or based on other conditions. In addition or in the alternative, drugs may be delivered from PRU (70) based on direct commands from a physician/clinician/nurse/etc., and a safety shell may simply restrict the delivery of drugs to the patient to ensure that the patient is not inadvertently overmedicated by the physician/clinician/nurse/etc. In addition or in the alternative, a safety shell may provide instructions to the physician/clinician/nurse/etc. regarding drug delivery and/or regarding the condition of the patient, based on data from BMU (40) and/or based on other conditions. Various suitable hardware components and firmware configurations that may be used to provide a safety shell control logic in PRU (70) will be apparent to those of ordinary skill in the art in view of the teachings herein. In the present example, the ultimate goal of a safety shell is to keep the patient safe.

Some versions of system (10) may be dedicated to use in certain medical procedures (e.g., colonoscopy and/or Esophagogastroduodenoscopy (EGD) procedures, etc.). For instance, a PRU (70) may be dedicated to a particular type of procedure, such that the safety shell control algorithm is relatively consistent each time PRU (70) is used. Some such versions of system (10) may thus include a relatively static set of safety shell control algorithms. However, some other versions of system (10) may be configured for use in various types of different medical procedures. In some such versions, the control logic carried out through a safety shell may vary based on the type of medical procedure in which system (10) will be used. For instance, the control logic may monitor different patient physiological parameters through BMU (40), based on the type of medical procedure in which system (10) will be used. In addition or in the alternative, the control logic may be responsive to different thresholds or trends in patient physiological parameters as detected through BMU (40), based on the type of medical procedure in which system (10) will be used. In addition or in the alternative, PRU (70) may vary the type, amount, timing, and/or duration, etc. of drug delivery based on the type of medical procedure in which system (10) will be used.

In versions where the safety shell control algorithm is adaptive based on the type of medical procedure in which system (10) will be used, there are various ways in which PRU (70) may be informed of the type of medical procedure in which system (10) will be used. In some such versions, the determination may be automated. For instance, the type of drug cassette (86) selected by a user may vary based on the medical procedure, and drug cassette (86) may include a barcode that is scanned by a reader coupled with PRU (70). PRU (70) may then process the reading from the barcode to automatically select the appropriate safety shell control algorithm or sub-algorithm. As another merely illustrative variation, drug cassette (86) may include an RFID chip or similar feature, and PRU (70) may include a reader associated with a slot that receives drug cassette (86). PRU (70) may process a reading from the RFID chip to automatically select the appropriate safety shell control algorithm or sub-algorithm. It should also be understood that PRU (70) may be manually informed of the type of medical procedure in which system (10) will be used. For instance, a user may make a selection via touch screen assembly (42), via touch screen assembly (72), via a computer device that is coupled with system (10) via a network, and/or via some other user input feature. Various other suitable ways in which system (10) may be informed of the type of medical procedure in which system (10) will be used will be apparent to those of ordinary skill in the art in view of the teachings herein. Furthermore, it should be understood that the safety shell control algorithm may be adaptive based on the type of patient (e.g., patient's physical sensitivity and/or known responsiveness to drugs, etc.) involved in the medical procedure. System (10) may be informed of the type of patient manually (e.g., via touchscreens (42, 72), etc.), based on data from a network, based on data from BMU (40), and/or otherwise.

It should also be understood that an adaptive safety shell control algorithm may be constructed in a nodal network fashion, with each node being associated with a single function of system (10) within the algorithm. Each node has a unique set of discrete, defined logic. This logic then has a communication aspect that communicates with the other nodes. The nodes in concert create a singular cohesive logical system. This may provide the ability to selectively enable/disable each node as well as modify a singular node, limiting a change or adaptation (prior to procedure or on the fly as assessed by the system) to that node. In some versions, parameter logic statements and actions may be defined by individual events with abstract relationships that provide actions/triggers. If a new parameter is required, the introduction of a nodal parameter/link relationship may provide a modified logic (e.g., instead of providing a new if/then nested set of logic, etc.). It may also be possible for nodes to be removed. Furthermore, concepts of fuzzy logic and/or neural networks may be employed within a nodal network type of control algorithm, allowing the control algorithm to handle case based ambiguity such as the relationship between different patient physiological parameters. It should thus be understood that a nodal network type of logic structure may provide significant flexibility, facilitating accommodation of different medical procedures and patients.

V. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

An apparatus comprising: (a) a device network server configured to store a set of device network data, the set of device network data comprising a patient identifier associated with a patient and a set of medical procedure data associated with a medical procedure to be performed on the patient; (b) a patient connector comprising a memory and a first communication device, wherein the patient connector is configured to be located proximate to the patient; and (c) a medical procedure device comprising a second communication device, wherein the medical procedure device is configured to be non-operable; wherein the memory is configured to store a subset of device network data, the subset of device network data comprising the patient identifier; wherein the patient connector is configured to send, via the first communication device, an identifying signal to the second communication device in response to a connection being established between the first communication device and the second communication device, wherein the identifying signal comprises the subset of device network data; wherein the second communication device is configured to receive the identifying signal; wherein the medical procedure device is configured to be operable based upon receiving the identifying signal via the second communication device.

### EXAMPLE 2

The apparatus of Example 1, wherein the first communication device and the second communication device each comprise one or more of: (i) an RFID device, (ii) a Bluetooth device, (iii) a Wi-Fi device, or (iv) a barcode scanner, or (v) a wired data connection device.

### EXAMPLE 3

The apparatus of any one or more of Examples 1 through 2, wherein the patient connector further comprises one or more of: (i) a patient monitor, (ii) a drug delivery connector, or (iii) a medication delivered manually and recorded, or (iv) a gas delivery connector; and wherein the medical procedure device comprises one or more of: (i) a bedside monitoring unit, (ii) a medical procedure room unit, or (iii) an automated responsiveness monitor.

### EXAMPLE 4

The apparatus of any one or more of Examples 1 through 3, wherein the subset of device network data further comprises a set of medical procedure data, and wherein the medical procedure device is further configured to configure itself with a set of operational parameters based upon the set of medical procedure data.

### EXAMPLE 5

The apparatus of Example 4, wherein the medical procedure device comprises a drug delivery device, and wherein the set of operational parameters comprise a safety shell control algorithm, and wherein the drug delivery device is configured to modify a drug delivery rate of a drug in response to the safety shell control algorithm.

### EXAMPLE 6

The apparatus of any one or more of Examples 1 through 5, wherein the first communication device and the second communication device are configured to establish the connection automatically based upon proximity between the first communication device and the second communication device, and wherein the first communication device and the second communication device each comprise a wireless communication device.

### EXAMPLE 7

The apparatus of any one or more of Examples 1 through 6, wherein the medical procedure device is further configured to request a set of medical procedure information from an interconnected device, wherein the patient identifier is included in the request, and wherein the interconnected comprises is one or more of: (i) the device network server, (ii) the patient connector, or (iii) a second medical procedure device.

### EXAMPLE 8

The apparatus of any one or more of Examples 1 through 7, wherein the subset of the set of device network data further comprises a set of user data, and wherein the medical procedure device is configured with a user profile, a set of user privileges, and a set of training record validations based upon the set of user data.

### EXAMPLE 9

The apparatus of any one or more of Examples 1 through 8, wherein the subset of device network data further comprises a device driver, wherein the device driver is configured to allow the medical procedure device to function with a second medical procedure device.

### EXAMPLE 10

The apparatus of any one or more of Examples 1 through 9, further comprising a second medical procedure device, wherein the second medical procedure device is in communication with the patient connector, and wherein the second medical procedure device is configured to communicate with the medical procedure device indirectly via the patient connector.

### EXAMPLE 11

The apparatus of any one or more of Examples 1 through 10, wherein the device network server is in communication with a patient record server, wherein the patient record server contains a set of patient data associated with the patient identifier, and wherein the device network server is configured to (a) receive a first subset of patient data from the patient server; and (b) transmit a second subset of patient data to the patient record server.

### EXAMPLE 12

A method comprising: (a) configuring a patient connector to receive a set of device network data from a device network server, the set of device network data comprising a patient identifier; (b) associating a patient with the patient connector, the patient connector comprising a memory and a first communication device; (c) connecting the patient connector with a medical procedure device via the first communication device; (d) sending the set of device network data to the medical procedure device via the patient connector; and (e) configuring the medical procedure device to prepare itself for a medical procedure based upon the patient identifier.

### EXAMPLE 13

The method of Example 12, wherein the set of device network data further comprises a set of medical procedure data, the method further comprising the step of configuring the medical procedure device to prepare itself with a set of operational parameters based upon the set of medical procedure data.

### EXAMPLE 14

The method of Example 13, wherein the medical procedure device comprises a drug delivery device, and wherein the set of operational parameters comprise a safety shell control algorithm, and wherein the drug delivery device modifies a drug delivery rate of a drug in response to the safety shell control algorithm.

### EXAMPLE 15

The method of any one or more of Examples 12 through 14, wherein connecting the patient connector with a medical procedure device comprises the steps of: (i) determining that the patient connector is proximate to the medical procedure device based upon a wireless signal communicated between the patient connector and the medical procedure device, and (ii) causing the patient connector to connect to the medical procedure device when they are proximate to each other.

### EXAMPLE 16

The method of any one or more of Examples 12 through 15, wherein the set of device network data further comprises a device driver, wherein the device driver is configured to allow the medical procedure device to function with a second medical procedure device, the method further comprising the step of configuring the medical procedure device with the device driver.

### EXAMPLE 17

The method of any one or more of Examples 12 through 16, further comprising the steps of: (a) connecting the patient connector to a second medical procedure device; (b) receiving a signal from the medical procedure device at the patient connector, the signal comprising an indication that the signal is intended for the second medical procedure device; and (c) sending the signal, via the patient connector, to the second medical procedure device.

### EXAMPLE 18

The method of any one or more of Examples 12 through 17, further comprising the steps of: (a) determining that the medical procedure device is not configured with a set of critical data that is needed for the medical procedure, the set of critical data comprising one or more of a device driver, a set of medical procedure info, or a set of user info; (b) configuring the medical procedure device to request the set of critical data from the device network server; (c) configuring the medical procedure device to, in response to an indication that the set of critical data is not available from the device network server, request the set of critical data from a local storage of the medical procedure device; and (d) configuring the medical procedure device to, in response to an indication that the set of critical data is not available from the local storage of the medical procedure device, request the set of critical data from a second medical procedure device.

### EXAMPLE 19

An apparatus comprising: (a) a patient connector comprising: (i) a memory, wherein the memory is configured to store a patient identifier, (ii) a data connector, and (iii) a medical device connector; (b) a pre medical procedure device comprising a pre medical procedure device connector, wherein the pre medical procedure device connector is adapted to couple with the medical device connector; (c) a medical procedure room unit comprising a medical procedure room unit connector, wherein the medical procedure room unit connector is adapted to couple with the medical device connector; and (d) a post medical procedure monitoring device comprising a monitor connector, wherein the monitor connector is adapted to couple with the medical device connector; wherein the pre medical procedure device is configured to receive, via the data connector, the patient identifier when the medical device connector is coupled with the pre medical procedure device connector; wherein the patient connector is configured to receive, from the pre medical procedure device, a set of pre medical procedure data associated with the patient identifier; wherein the medical procedure room unit is configured to receive, via the data connector, the patient identifier and the set of pre medical procedure data when the medical device connector is coupled with the medical procedure room unit connector, wherein the medical procedure room unit is automatically configured for a medical procedure based upon the patient identifier and the set of pre medical procedure data; wherein the patient connector is configured to receive, from the medical procedure room unit, a set of medical procedure data associated with the patient identifier; and wherein the post medical procedure monitoring device is configured to receive, via the data connector, the patient identifier, the set of pre medical procedure data, and the set of medical procedure data when the medical device connector is coupled with the monitor connector, wherein the post medical procedure monitoring device is automatically configured for monitoring a patient based upon the patient identifier, the set of pre medical procedure data, and the set of medical procedure data.

### EXAMPLE 20

The apparatus of Example 19, wherein the data connector and the medical device connector are combined into a single connector; wherein the pre medical procedure device comprises two or more of: (i) an arterial oxygen saturation monitor, (ii) a non invasive blood pressure monitor, (iii) an automated responsiveness monitor, or (iv) a communication device connected to a medical record server; wherein the medical procedure room unit comprises a drug delivery device and an oxygen delivery device; and wherein the memory comprises a removable digital memory card.

VI. Miscellaneous

It should be understood that any of the examples described herein may include various other features in addition to or in lieu of those described above. By way of example only, any of the devices herein may also include one or more of the various features disclosed in any of the various references that are incorporated by reference herein. It should also be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

It should also be understood that any ranges of values referred to herein should be read to include the upper and lower boundaries of such ranges. For instance, a range expressed as ranging “between approximately 1.0 inches and approximately 1.5 inches” should be read to include approximately 1.0 inches and approximately 1.5 inches, in addition to including the values between those upper and lower boundaries.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, Calif. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a device network server configured to store a set of device network data, the set of device network data comprising a patient identifier associated with a patient and a set of medical procedure data associated with a medical procedure to be performed on the patient;
(b) a patient connector comprising a memory and a first communication device, wherein the patient connector is configured to be located proximate to the patient; and
(c) a medical procedure device comprising a second communication device, wherein the medical procedure device is configured to be non-operable;
wherein the memory is configured to store a subset of device network data, the subset of device network data comprising the patient identifier;
wherein the patient connector is configured to send, via the first communication device, an identifying signal to the second communication device in response to a connection being established between the first communication device and the second communication device, wherein the identifying signal comprises the subset of device network data;
wherein the second communication device is configured to receive the identifying signal;
wherein the medical procedure device is configured to be operable based upon receiving the identifying signal via the second communication device.

2. The apparatus of claim 1, wherein the first communication device and the second communication device each comprise one or more of:
(i) an RFID device,
(ii) a Bluetooth device,
(iii) a Wi-Fi device,
(iv) a barcode scanner, or
(v) a wired data connection device.

3. The apparatus of claim 1, wherein the patient connector further comprises one or more of:
(i) a patient monitor,
(ii) a drug delivery connector,
(iii) a medication delivered manually and recorded, or
(iv) a gas delivery connector; and
wherein the medical procedure device comprises one or more of:
(i) a bedside monitoring unit,
(ii) a medical procedure room unit, or
(iii) an automated responsiveness monitor.

4. The apparatus of claim 1, wherein the subset of device network data further comprises a set of medical procedure data, and wherein the medical procedure device is further configured to configure itself with a set of operational parameters based upon the set of medical procedure data.

5. The apparatus of claim 4, wherein the medical procedure device comprises a drug delivery device, and wherein the set of operational parameters comprise a safety shell control algorithm, and wherein the drug delivery device is configured to modify a drug delivery rate of a drug in response to the safety shell control algorithm.

6. The apparatus of claim 1, wherein the first communication device and the second communication device are configured to establish the connection automatically based upon proximity between the first communication device and the second communication device, and wherein the first communication device and the second communication device each comprise a wireless communication device.

7. The apparatus of claim 1, wherein the medical procedure device is further configured to request a set of medical procedure information from an interconnected device, wherein the patient identifier is included in the request, and wherein the interconnected comprises is one or more of:
(i) the device network server,
(ii) the patient connector, or
(iii) a second medical procedure device.

8. The apparatus of claim 1, wherein the subset of the set of device network data further comprises a set of user data, and wherein the medical procedure device is configured with a user profile, a set of user privileges, and a set of training record validations based upon the set of user data.

9. The apparatus of claim 1, wherein the subset of device network data further comprises a device driver, wherein the device driver is configured to allow the medical procedure device to function with a second medical procedure device.

10. The apparatus of claim 1, further comprising a second medical procedure device, wherein the second medical procedure device is in communication with the patient connector, and wherein the second medical procedure device is configured to communicate with the medical procedure device indirectly via the patient connector.

11. The apparatus of claim 1, wherein the device network server is in communication with a patient record server, wherein the patient record server contains a set of patient data associated with the patient identifier, and wherein the device network server is configured to:
(a) receive a first subset of patient data from the patient record server; and
(b) transmit a second subset of patient data to the patient record server.

12. A method comprising:
(a) configuring a patient connector to receive a set of device network data from a device network server, the set of device network data comprising a patient identifier;
(b) associating a patient with the patient connector, the patient connector comprising a memory and a first communication device;
(c) connecting the patient connector with a medical procedure device via the first communication device;
(d) sending the set of device network data to the medical procedure device via the patient connector; and
(e) configuring the medical procedure device to prepare itself for a medical procedure based upon the patient identifier.

13. The method of claim 12, wherein the set of device network data further comprises a set of medical procedure data, the method further comprising the step of configuring the medical procedure device to prepare itself with a set of operational parameters based upon the set of medical procedure data.

14. The method of claim 13, wherein the medical procedure device comprises a drug delivery device, and wherein the set of operational parameters comprise a safety shell control algorithm, and wherein the drug delivery device modifies a drug delivery rate of a drug in response to the safety shell control algorithm.

15. The method of claim 12, wherein connecting the patient connector with a medical procedure device comprises the steps of:
(i) determining that the patient connector is proximate to the medical procedure device based upon a wireless signal communicated between the patient connector and the medical procedure device, and
(ii) causing the patient connector to connect to the medical procedure device when they are proximate to each other.

16. The method of claim 12, wherein the set of device network data further comprises a device driver, wherein the device driver is configured to allow the medical procedure device to function with a second medical procedure device, the method further comprising the step of configuring the medical procedure device with the device driver.

17. The method of claim 12, further comprising the steps of:
(a) connecting the patient connector to a second medical procedure device;
(b) receiving a signal from the medical procedure device at the patient connector, the signal comprising an indication that the signal is intended for the second medical procedure device; and
(c) sending the signal, via the patient connector, to the second medical procedure device.

18. The method of claim 12, further comprising the steps of:
(a) determining that the medical procedure device is not configured with a set of critical data that is needed for the medical procedure, the set of critical data comprising one or more of a device driver, a set of medical procedure info, or a set of user info;
(b) configuring the medical procedure device to request the set of critical data from the device network server;
(c) configuring the medical procedure device to, in response to an indication that the set of critical data is not available from the device network server, request the set of critical data from a local storage of the medical procedure device; and
(d) configuring the medical procedure device to, in response to an indication that the set of critical data is not available from the local storage of the medical procedure device, request the set of critical data from a second medical procedure device.

19. An apparatus comprising:
(a) a patient connector comprising:
(i) a memory, wherein the memory is configured to store a patient identifier,
(ii) a data connector, and
(iii) a medical device connector;
(b) a pre medical procedure device comprising a pre medical procedure device connector, wherein the pre medical procedure device connector is adapted to couple with the medical device connector;
(c) a medical procedure room unit comprising a medical procedure room unit connector, wherein the medical procedure room unit connector is adapted to couple with the medical device connector; and
(d) a post medical procedure monitoring device comprising a monitor connector, wherein the monitor connector is adapted to couple with the medical device connector;
wherein the pre medical procedure device is configured to receive, via the data connector, the patient identifier when the medical device connector is coupled with the pre medical procedure device connector;
wherein the patient connector is configured to receive, from the pre medical procedure device, a set of pre medical procedure data associated with the patient identifier;
wherein the medical procedure room unit is configured to receive, via the data connector, the patient identifier and the set of pre medical procedure data when the medical device connector is coupled with the medical procedure room unit connector, wherein the medical procedure room unit is automatically configured for a medical procedure based upon the patient identifier and the set of pre medical procedure data;
wherein the patient connector is configured to receive, from the medical procedure room unit, a set of medical procedure data associated with the patient identifier; and
wherein the post medical procedure monitoring device is configured to receive, via the data connector, the patient identifier, the set of pre medical procedure data, and the set of medical procedure data when the medical device connector is coupled with the monitor connector, wherein the post medical procedure monitoring device is automatically configured for monitoring a patient based upon the patient identifier, the set of pre medical procedure data, and the set of medical procedure data.

20. The apparatus of claim 19, wherein the data connector and the medical device connector are combined into a single connector;
wherein the pre medical procedure device comprises two or more of:
(i) an arterial oxygen saturation monitor,
(ii) a non invasive blood pressure monitor,
(iii) an automated responsiveness monitor, or
(iv) a communication device connected to a medical record server;
wherein the medical procedure room unit comprises a drug delivery device and an oxygen delivery device; and
wherein the memory comprises a removable digital memory card.
